(19) United States
(12) Patent Application Publication
(10) Pub. No.: US 2024/0074707 A1
(43) Pub. Date: Mar. 7, 2024

(54) ORACLE - A PHM TEST & VALIDATION PLATFORM FOR ANOMALY DETECTION IN BIOTIC OR ABIOTIC SENSOR DATA

(71) Applicant: Arizona Board of Regents on Behalf of the University of Arizona, Tucson, AZ (US)

(72) Inventor: Wolfgang Fink, Tucson, AZ (US)

(21) Appl. No.: 18/280,395

(22) PCT Filed: Mar. 4, 2022
(86) PCT No.: PCT/US2022/070992

§ 371 Date: Sep. 5, 2023

Related U.S. Application Data
(60) Provisional application No. 63/156,416, filed on Mar. 4, 2021.

(51) Int. Cl. A61B 5/00; A61B 5/318; G06N 3/084; G16H 40/63

(52) U.S. Cl. CPC A61B 5/7221; A61B 5/318; A61B 5/7267; G06N 3/084; G16H 40/63; A61B 2560/0242

(57) ABSTRACT

Various examples are provided related to anomaly detection in sensor data (e.g., biotic or abiotic sensor data). In one example, a method includes applying data from portions of a real-time sensor signal to an artificial neural network trained to identify motifs associated with the real-time sensor signal; detecting a transition from a first motif to a second motif based upon changes in output signals providing an indication of correlation of the sensor signal to the motifs; and identifying a change in an environmental condition based upon the transition between the motifs. In another example, a method includes selecting a plurality of motifs associated with a desired training signal; generating the desired training signal by transitioning between different motifs in a pseudo-random basis; and generating training data sets from the desired training signal, which can then be utilized to train a network or other machine learning system.

### CROSS REFERENCE TO RELATED APPLICATIONS

This application claims priority to, and the benefit of, co-pending U.S. provisional application entitled “ORACLE—A PHM Test & Validation Platform for Anomaly Detection in Biotic of Abiotic Sensor Data” having Ser. No. 63/156,416, filed Mar. 4, 2021, which is hereby incorporated by reference in its entirety.

### STATEMENT REGARDING FEDERALLY SPONSORED RESEARCH OR DEVELOPMENT

This invention was made with government support under Grant No. 80NSSC19C0130, awarded by NASA. The government has certain rights in the invention.

### BACKGROUND

For manned space exploration missions beyond Low Earth Orbit (LEO), regular resupply of consumables, delivery of new supplies and replacement components, as well as emergency quick-return options may not in general be easy, timely, or feasible. Success in such space missions requires solutions to difficult technical challenges, built on proven capabilities, which may require the development of new capabilities arising from the development of novel cutting-edge technologies.

Prognostics and Health Management (PHM) is an engineering discipline that focuses on the fundamental principles of system failures to predict mean time between failures (MTBF) and links the principles to system life cycle management. PHM-based technology can provide early warning of failure and assess the potential for life extension.

### SUMMARY

Aspects of the present disclosure are related to anomaly detection in sensor data (e.g., biotic or abiotic sensor data). In one aspect, among others, a method for anomaly detection in sensor data comprises applying data from portions of a real-time sensor signal to an artificial neural network trained to identify a plurality of motifs associated with the real-time sensor signal based upon corresponding output signals of the artificial neural network the corresponding output signals providing an indication of correlation of the real-time sensor signal to each of the plurality of motifs; detecting a transition from a first motif of the plurality of motifs to a second motif of the plurality of motifs based upon changes in a first output signal corresponding to the first motif and a second output signal corresponding to the second motif; and identifying a change in an environmental condition based upon the transition between the first and second motifs.

In one or more aspects, the artificial neural network can be trained using error-backpropagation or a stochastic training approach. The artificial neural network can be a multilayer feedforward network, a deep learning network, a convolutional neural network, or a recursive neural network. The artificial neural network can be trained using error-backpropagation or a stochastic training approach. The artificial neural network can be trained using data generated from previously extracted motifs associated with the real-time sensor signal. The generated data can comprise transitions between two of the previously extracted motifs. The generated data can comprise superimposed noise. At least one of the previously extracted motifs can comprise superimposed noise. The previously extracted motifs can be generated by averaging raw sensor signal data and binning into a histogram. In various aspects, the real-time sensor signal can be a biotic signal. The biotic signal can be a vital sign of an individual exposed to the environmental condition. The biotic signal can be an electrocardiogram (ECG) signal. The environmental condition can be associated with a habitat of the individual. The real-time sensor signal can be an abiotic signal. The biotic signal can be a vital sign of an individual exposed to the environmental condition. The biotic signal can be an electrocardiogram (ECG) signal, blood oxygenation, EEGs (brain waves), temperature, ocular structure changes, or visual field changes. The environmental condition can be associated with a habitat of the individual.

In another aspect, a method for generating training data for training a network comprises selecting a plurality of motifs associated with a desired training signal; generating the desired training signal by transitioning between different motifs of the plurality of motifs in a pseudo-random basis; and generating training data sets from the desired training signal. In one or more aspects, the transitions between different motifs comprise nonlinear transitions. Generating the desired training signal can further comprise superimposing noise. The plurality of motifs can be generated by averaging raw sensor signal data and binning into a histogram. The method can further comprising training an artificial neural network using at least a portion of the generated training data sets.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D illustrate examples of extracted ECG motifs including apnea, ventricular tachyarrhythmia, supraventricular arrhythmia, and normal, respectively, in accordance with various embodiments of the present disclosure.

FIG. 2 illustrates an example of ECG signal morphing, in accordance with various embodiments of the present disclosure.

FIG. 3 illustrates an example of ECG signal morphing across user-defined motifs, in accordance with various embodiments of the present disclosure.

FIG. 4 illustrates an ORACLE Visualizer displaying examples of ECG data without and with Gaussian noise, in accordance with various embodiments of the present disclosure.

FIG. 5 illustrates the ORACLE Visualizer displaying an example of ECG motif morphing in progress, in accordance with various embodiments of the present disclosure.

FIG. 6. Illustrates an example of time-stamped data output of ORACLE, in accordance with various embodiments of the present disclosure.

FIG. 7A illustrates an example of the general architecture of a multi-layer feedforward network and FIG. 7B illustrates an example of the actual network irnplementation, in accordance with various embodiments of the present disclosure.

FIG. 8 illustrates an example of time development of the training error behavior for the network of FIG. 7B, in accordance with various embodiments of the present disclosure.

FIG. 9 illustrates an example of the ORACLE Visualizer displaying anomalies based upon the four motifs of FIGS. 1A-1D, in accordance with various embodiments of the present disclosure.

FIG. 10 illustrates an example of single ECG motif and associated ECG raw data streams generated by the ORACLE without any added noise, in accordance with various embodiments of the present disclosure.

FIGS. 11 and 12 illustrate examples of classification of motif M2 in the presence of noise, in accordance with various embodiments of the present disclosure.

FIG. 13 illustrates an example of classification of transition between motif M4 and motif M3 in the absence of noise, in accordance with various embodiments of the present disclosure.

FIG. 14 illustrates an example of classification of transition between motif and motif M2 in the presence of noise, in accordance with various embodiments of the present disclosure.

FIG. 15 illustrates an example of classification of transition between motif M1 and motif M4 in the presence of noise, in accordance with various embodiments of the present disclosure.

FIG. 16 illustrates an example of classification of transition between motif M4 and motif M2 in the presence of noise, in accordance with various embodiments of the present disclosure.

FIG. 17 is a schematic block diagram illustrating an example of a system employed for the ORACLE methodology, in accordance with various embodiments of the present disclosure

### DETAILED DESCRIPTION

Disclosed herein are various examples related to anomaly detection in sensor data (e.g., biotic or abiotic sensor data). Methods, systems and apparatus for collection and processing of biotic or abiotic sensor data are presented. Abiotic sensors aboard the International Space Station (ISS), such as the Volatile Organic Analyzer (VOA), utilize gas chromatography and ion mobility spectrometry to detect and monitor harmful gasses and chemicals that pose serious risks to individuals in closed environments. Utilizing individuals as biosensors to complement abiotic sensor arrays provides autonomous systems a more comprehensive amount of data to interpret, ensuring the health and safety of everyone aboard. Reference will now be made in detail to the description of the embodiments as illustrated in the drawings, wherein like reference numbers indicate like parts throughout the several views.

Utilizing electrocardiogram (ECG) data as an example of a non-invasively obtained vital sign (which is equally applicable to abiotic sensor data), a PHM test & validation platform—the ORACLE—has been devised that can auto-generate raw ECG data from previously extracted ECG motifs, ultimately representative of various conditions, e.g., health conditions, resulting from space habitat environment changes. The ORACLE emulates the occurrence of environmental changes and generates a log file of the ground truth, i.e., how the occurrence of environmental changes is emulated, to allow for training and validation of subsequent deep learning-based anomaly detection frameworks, serving as a proof of concept for integration as a subsystem into a novel overarching PHM-based anomaly detection framework for monitoring space habitat health.

The ORACLE can be equipped with an anomaly detection framework using artificial neural networks, such as, but not limited to, multi-layer feedforward networks, deep learning networks, convolutional neural networks, recurrent neural networks, artificial neural networks, or other deep learning or neural network approaches. The measured “biometric” changes in each crew member can be utilized as a supplementary indicator to detect degradation in the space habitat environment and associated habitat subsystems. This novel indicator has the potential to enhance existing capabilities by providing for earlier detection than might otherwise be possible. It also has the potential to provide detection that only a plurality or ensemble of heterogeneous abiotic sensors, when integrated or looked at in complex ways, may or may not be able to deliver. The general idea is to use the crew health status as an integrated contributor to other PHM capabilities already implemented on associated space habitat subsystems.

With NASA's push for prolonged stays aboard orbiting or surface-based space habitats (e.g., NASA Gateway/Artemis programs and successors) on the Moon and soon on Mars, there is a need for continuous monitoring of life support and mission critical systems deployed on these space habitats. Abiotic sensors may be used to detect anomalies aboard these space habitats, which are expected to also be embedded in a Prognostics and Health Management (PHM) framework. A crew member can be employed as a biosensor to be integrated into an overarching space habitat PHM system to increase the breadth of available data when monitoring environment health. Utilizing electrocardiogram (ECG) data as an example of a non-invasively obtained vital sign, a PHM test & validation platform—the ORACLE—has been devised that auto-generates raw ECG data from previously extracted ECG motifs, ultimately representative of various conditions resulting from environment changes aboard a space habitat.

The ORACLE emulates the occurrence of environmental changes and generates a log file to allow for training and validation of subsequent deep learning-based anomaly detection frameworks, serving as a proof of concept for integration as a subsystem into a novel overarching PHM-based anomaly detection framework for monitoring space habitat health. Moreover, the ORACLE can be equipped with an anomaly detection framework using artificial neural networks, such as, but not limited to, multi-layer feedforward networks, deep learning networks, convolutional neural networks, recurrent neural networks, or other deep learning or neural network approaches. This disclosure showcases an example of the workings of the ORACLE and presents preliminary results for the deep learning-based anomaly detection framework using a multi-layer feedforward network (or multiplayer perceptron) as a stand-in for other artificial neural networks or deep learning approaches. The measured “biometric” changes in each crew member can be used as a supplementary indicator to detect degradation or changes in the space habitat environment and/or associated habitat subsystems. This novel indicator has the potential to enhance existing capabilities by providing for earlier detection than might otherwise be possible. Monitoring one or more crew member can deliver an indication for an otherwise very complex situation that would otherwise need evaluation of multiple heterogeneous abiotic sensors to detect. The crew health status can be used as an integrated contributor to other PHM capabilities already implemented on associated space habitat subsystems.

### Introduction

NASA is making rapid progress towards a more sustained presence aboard orbiting or surface-based space habitats (e.g., NASA Gateway/Artemis programs and successors) on the Moon and soon on Mars. As such, there is a need for continuous monitoring of life support and mission critical systems deployed on these space habitats, both in the presence and absence of crew members, to ensure a healthy/viable onboard environment. There will invariably be a heavy reliance on abiotic sensors to detect anomalies aboard these space habitats. Based upon this assumption, a crew member may be used as a biosensor to be integrated into an overarching space habitat PHM framework. This biomimetic concept is akin to the “canary in the coal mine,” informing environmental changes aboard space habitats due to events, such as outgassing events, oxygen leaks, etc. The measured “biometric” changes in each crew member can be used as a supplemental indicator of early degradation in the space habitat environment and associated habitat subsystems, which may enhance existing detection capabilities. The crew health status can be used as an integrated contributor to other PHM capabilities already implemented on associated space habitat subsystems.

### Synopsis

Choosing electrocardiogram (ECG) data as a mode of noninvasively obtained vital signs, a Prognostics and Health Management (PHM) test & validation platform—termed the ORACLE—was devised that auto-aenerates raw ECG data from previously extracted ECG motifs, representative of various conditions (e.g., health conditions), and super-imposes noise to the data to simulate sub-optimal/imperfect sensor readings. Moreover, the ORACLE is capable of transitioning from one ECG motif to another on a pseudo-random basis to emulate and/or simulate the occurrence of changes in the environment of a space habitat or other isolated environment (e.g., submarines, mines, aircraft, etc.) that can influence the vital signs of individuals (e.g., crew members) in that changed environment setting.

The associated ORACLE Visualizer can display the auto-generation of ECG raw data onscreen in real time. Furthermore, the ORACLE can create on demand, a time-stamped log file that records the entire ECG raw data auto-generation. This log file can serve as a “ground truth” for both training and validating subsequent deep learning-based anomaly detection frameworks.

The ORACLE can utilize an artificial neural network or other deep learning approach to detect the onset and/or presence of various ECG motifs (both normal and/or anomalous ones) with or without noise in real time in the ECG data stream generated by the ORACLE. A user-controllable degree of, e.g., Gaussian sensor noise serves as a means to quantify the breakdown of any employed anomaly detection framework, which is important to determine its respective robustness.

The ORACLE can be operated as a standalone application or can be queried as an embeddable dynamically linked library module for integration as a subsystem into an overarching PHM-based anomaly detection framework for space habitat health. The workings of the ORACLE and associated ORACLE Visualizer are described, and preliminary performance results of the artificial neural network-based ORACLE anomaly detection framework are presented.

“The ORACLE” gleams its name from Greek mythology, specifically the Oracle of Delphi. In terms of function, the namesake of the ORACLE platform relates to the mythological process of divination of the future from the Oracle of Delphi. The ORACLE can be queried, and ECG motif data returned at the output, while the inner workings are unbeknownst to the user.

### Motif Extraction

A method to extract ECG motifs based on intrasubject averaging of R-R interval ECG raw data and binning into a histogram with a user-defined (or, alternatively, recorded) frequency resolution for certain individual heart conditions of patients was devised using ECG raw data from the Physionet ECG Database, Physionet Apnea-ECG Database, and Creighton University Ventricular Tachyarrhythmia database. The ECG motifs serve as an “average” representative of a recorded ECG signal for a given period, encompassing the most prominent features and variations recorded by an ECG in an R-R interval. Using a finite, prerecorded ECG data file with annotated R peaks from the Physionet Database, it is possible to develop these motif structures. Starting with calculating the average R-R interval for the raw ECG dataset, a histogram with a user-defined number of bins can be mapped to subdivide the calculated average R-R interval into equal bin ranges. For example, if the average R-R interval is 1 second, and the user-defined number of bins is 100, the mapped histogram spans from 0 to 1 second, with each bin spanning 10 ms.

With the newly created histogram spanning the length of the average R-R interval, the process of tabulating the ECG values in an R-R interval of the raw dataset can be started to generate the motif. Using the first R peak of an arbitrary R-R interval in the dataset with its timestamp set as the new starting point when mapped to the histogram (i.e., falls into the first bin), the ECG value at that index can be added to the histogram bin, Continuing until the end of the specific R-R interval (i.e., the next R peak is reached), each discrete value of the raw ECG can be assigned to the respective histogram bin.

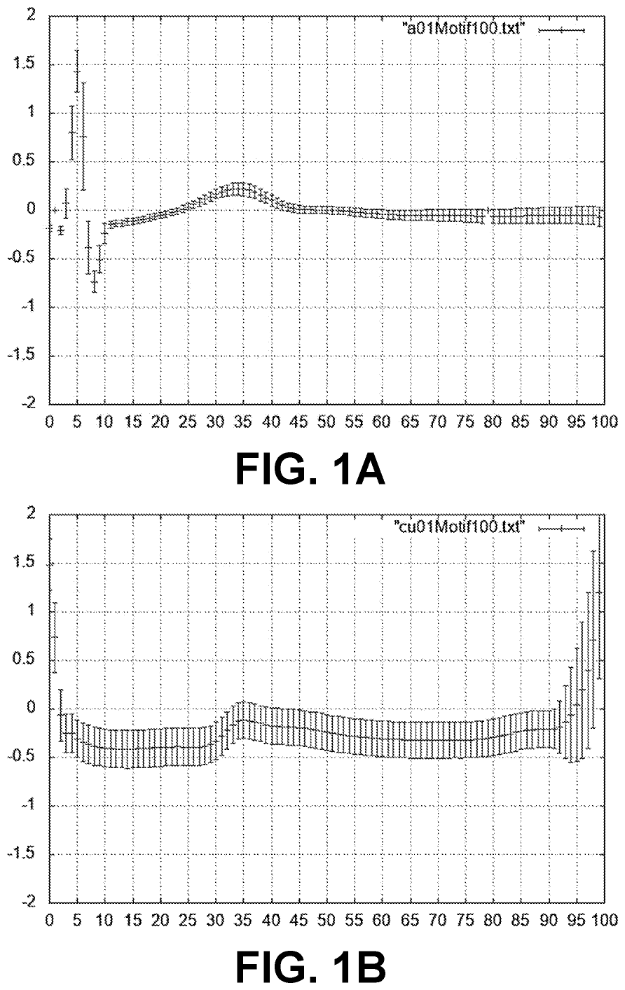

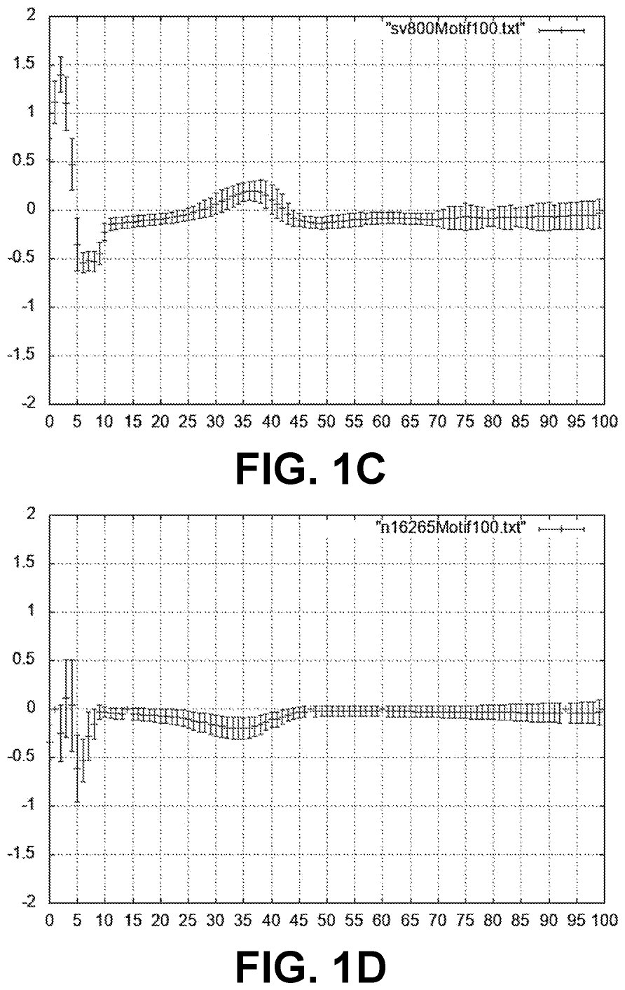

Following this process for the entire dataset, at the end, each bin can be averaged and the standard deviation can be calculated for the user-defined number of bins, which represent the underlying motif structure. FIGS. 1A-1D illustrate examples of extracted ECG motifs including apnea, ventricular tachyarrhythmia, supraventricular arrhythmia, and normal, respectively. Note that for R-R intervals that exceed the average R-R interval length (i.e., ECG values that fall outside the range of the histogram bins), these ECG values are incorporated into the last bin of the histogram regardless. This may lead to R-R interval duration artifacts, which may have to be addressed at a later point.

The preliminary inter-subject comparison of respectively extracted motifs points towards high inter-subject variability. However, it should be noted that the inter-subject variability does not pose a problem necessarily as motifs would be established individually for each crew member, and deviations in each, due to environmental changes aboard a space habitat, would be monitored respectively. An alternate method to the generation of a motif is to simply cast a raw R-R interval or other raw interval of the SQRT complex in the raw ECG data into a histogram with a user-defined number of bins. This approach may have advantages over the averaged motif generation as certain patient or crew member specific nuances of the ECG data may be preserved and not averaged out.

### Motif Morphing

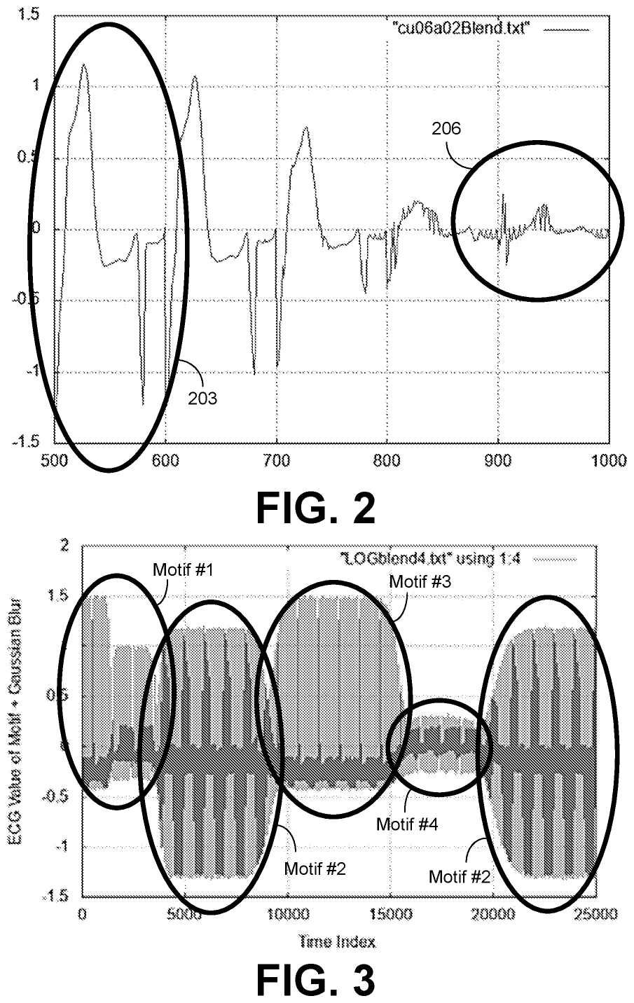

To lay the foundation for a crew member vital sign ECG simulator, a first prototype was developed that permits ECG signal morphing from one ECG-state of a crew member to another ECG-state so as to simulate the change in ECG signals of a crew member in response to an environmental change aboard a space habitat. FIG. 2 illustrates an example of ECG signal morphing from one ECG-state/motif (circumscribed with ellipse 203 on the left) of a crew member to another ECG-statelmotif (circumscribed with ellipse 206 on the right) using a sigmoidal transition function.

### ORACLE Description

Choosing electrocardiogram (ECG) data as a representative example of non-invasively obtained vital signs, such as blood oxygenation or electroencephalogram (EEG) data, a test and validation platform—the ORACLE—was devised that auto-generates raw ECG data from previously extracted ECG motifs, representative of various conditions that could ultimately be caused by environmental changes aboard, e.g., a space habitat. FIG. 3 illustrates an example of ECG signal morphing across previously extracted motifs, ultimately representative of various health conditions due to environment changes aboard a space habitat.

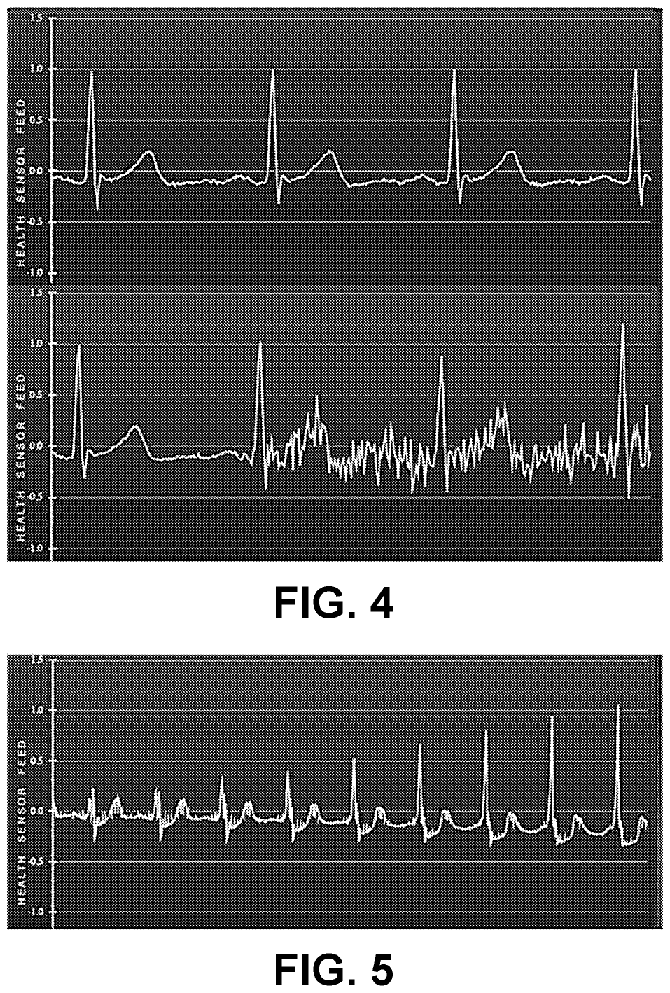

Associated with the ORACLE ECG raw data auto-generator, an ORACLE Visualizer was devised (see, e.g., FIGS. 4, 5 and 9-14) that displays the auto-generation of ECG raw data onscreen in real time. The top image of FIG. 4 shows an ORACLE Visualizer displaying an example of ECG data without Gaussian noise being added. The ORACLE can super-impose Gaussian noise randomly and in real time to the data according to a user-defined degree to simulate suboptimal; imperfect sensor readings. The bottom image of FIG. 4 shows an ORACLE Visualizer displaying an example of ECG data with Gaussian noise added to simulate sub-optimal/imperfect sensor readings. The user-controllable degree of Gaussian sensor noise serves as a means to quantify the breakdown of any employed anomaly detection framework which is important to determine its respective robustness.

The ORACLE is capable of morphing from one ECG motif to another on a pseudo-random basis within a user-defined maximum and minimum transition time period using different transition functions, e.g., sigmoidal, linear, exponential, logarithmic, etc. The image of FIG. 5 shows the ORACLE Visualizer displaying an example of ECG Motif morphing in progress. The process of morphing between motifs emulate/simulates the occurrence of changes in the environment of a space habitat, which influences the vital signs of a crew member. Once transitioned into a new ECG motif, the ORACLE remains/dwells in that motif for a pseudo-random period of time, again within a user-defined maximum and minimum time period.

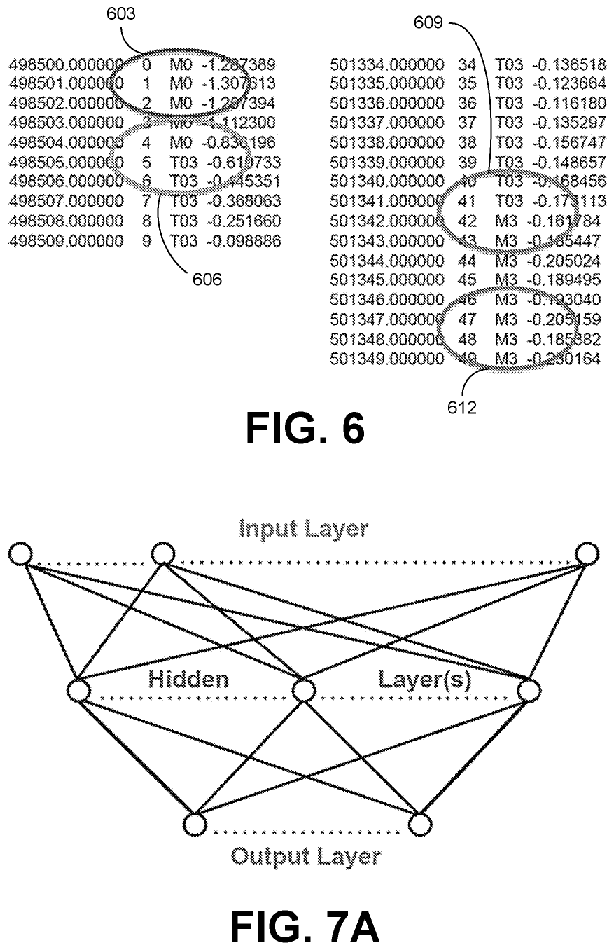

Moreover, the ORACLE can be equipped with the ability to generate on demand a time-stamped log file that records the “ground truth” underlying the entire ECG raw data auto-generation, with and/or without Gaussian noise applied, which motif is active at any time, and the ECG motif rnorphing processes. This log file is key for both training and validating subsequent deep learning-based anomaly detection frameworks. FIG. 6. illustrates an example of time-stamped data output of ORACLE as “Ground Truth” for subsequent anomaly detection via, e.g. ML/DL algorithms. Ellipse 603 shows the currently active motif M0, ellipse 606 shows the start of the transition/morphing between motif M0 and motif M3, ellipse 609 shows the end of the transition/morphing between motif M0 and motif M3, and ellipse 612 shows the now active motif M3.

### ORACLE Anomaly Detection

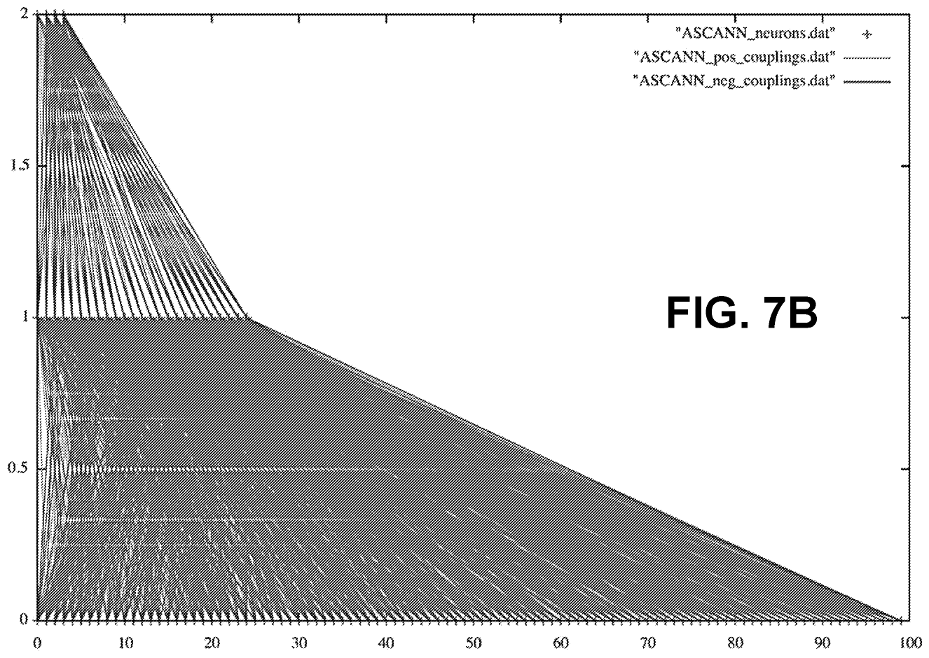

The ORACLE currently employs multi-layer feedforward networks (FFNs) or multi-layer perceptrons as a stand-in for other artificial neural networks or deep neural networks with an input layer, one or several hidden layers, and an output layer for the anomaly detection in real time. FIG. 7A illustrates an example of the general architecture of a multi-layer feedforward network (FFN), i.e., multi-layer perceptron. FIG. 7B illustrates an example of the actual network implementation used in the anomaly detection framework: FFN with 100 input units, one hidden layer with 25 hidden units, and 4 output units. The neurons are, e.g., of McCulloch Pitts type. Note that in FIG. 7B the input layer is on the bottom and the output layer on the top, and the network includes both positive couplings and negative couplings. The output of the anomaly detection (artificial neural network in general, and multi-layer perceptron in particular) can be used without a window-averaging approach, i.e., potentially spiking data, or smoothened out by using a window-averaging approach of the anomaly detection output.

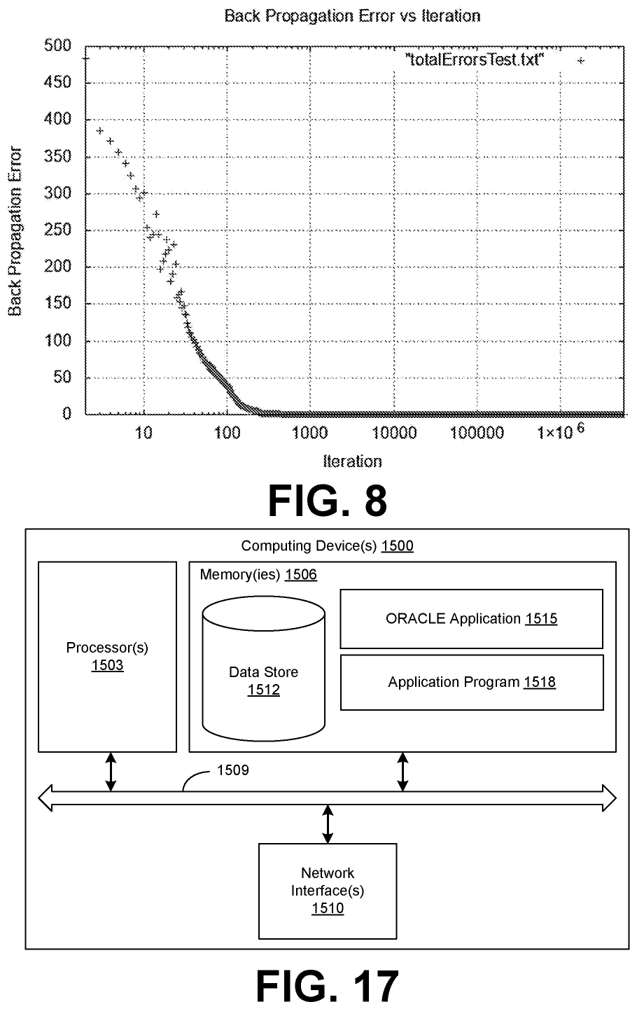

The update equation of every neuron within a multi-layer perceptron employed is:

$${S}_{j} = f ( {\sum }_{i = 1}^{N} {J}_{ij} {S}_{i} - {θ}_{j} ) ,$$

where Sⱼ denotes the state of hidden/output neuron j, Sᵢ the state of input/hidden neuron i, Jᵢⱼ the neural coupling strength between input/hidden neuron Sᵢ and hidden/output neuron Sⱼ, and ϑⱼ the threshold for hidden/output neuron Sⱼ. The ƒ(x) function is the transfer function, in this case: 1/(1+e^{-αx}). For the training of the multi-layer feedforvvard networks error-backpropagation was used, Alternatively, a stochastic training approach can be used (see, e.g., Fink W (2009) Autonomous Self-Configuration of Artificial Neural Networks for Data Classification or System Control; Proc. SPE, Vol. 7331, 733105 (2009); DOI:10.1117/12.821836). FIG. 8 shows the time development of the training error behavior as a function of error-backpropagation iterations for the multi-layer perceptron depicted in FIG. 7B.

### ORACLE Preliminary Anomaly Detection Performance

In the following preliminary results are presented for the anomaly detection within ORACLE based on a multi-layer perceptron with one hidden layer of 25 hidden units. In this case, an anomaly is defined as the deviation of the ECG data, coming from the ORACLE (i.e., as a stand-in for the actual individual crew member aboard a space habitat), from a normal ECG motif (FIG. 1D): this means the onset of one of the ECG motifs associated with a health condition (FIGS. 1A-1C) that may be the result of a change in the environment aboard a space habitat.

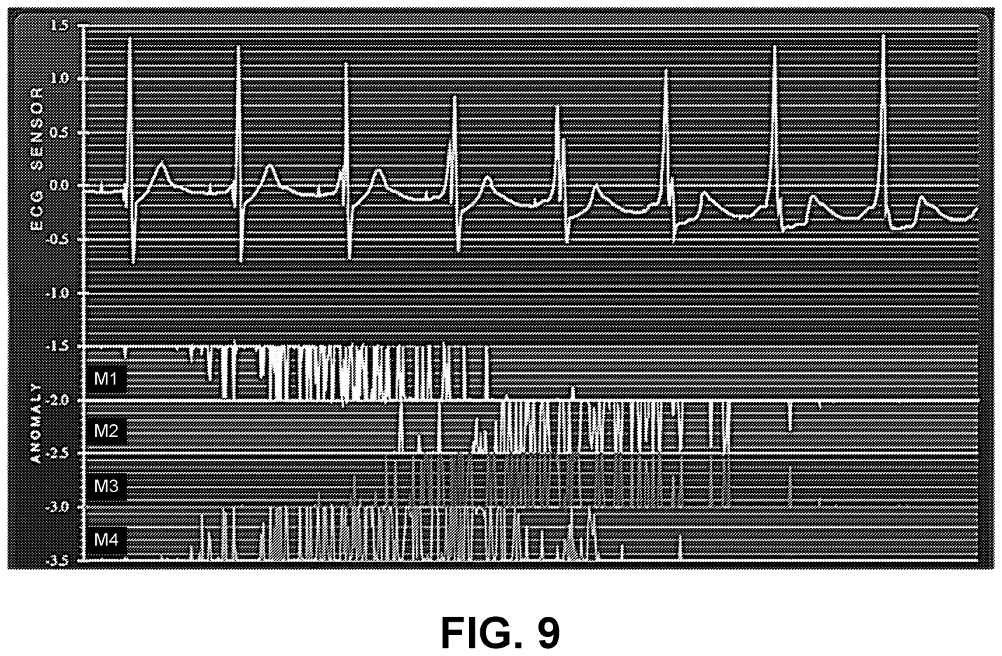

Referring to FIG. 9, the anomalies are shown as 4 curves on the bottom of the expanded/revised ORACLE Visualizer that run concurrently along the ECG data stream generated by the ORACLE randomly engaging one of four motifs (FIGS. 1A-1D) with randomly selected dwell times and transition times—all of this being captured in a log file (FIG. 6). The 4 bottom curves indicate concurrently which motif is currently active based on the built-in anomaly detection in ORACLE.

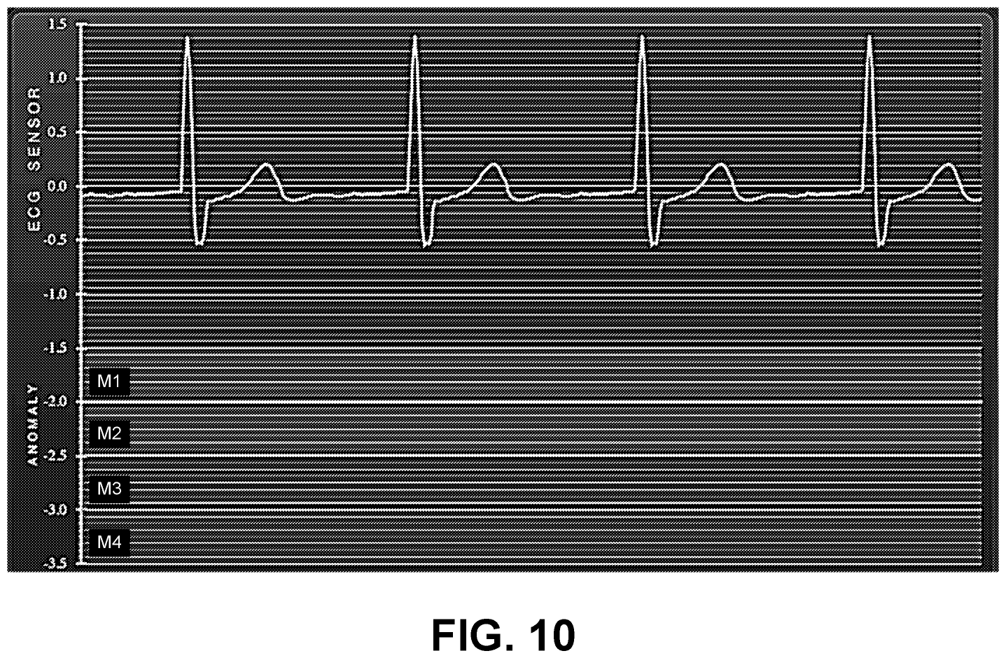

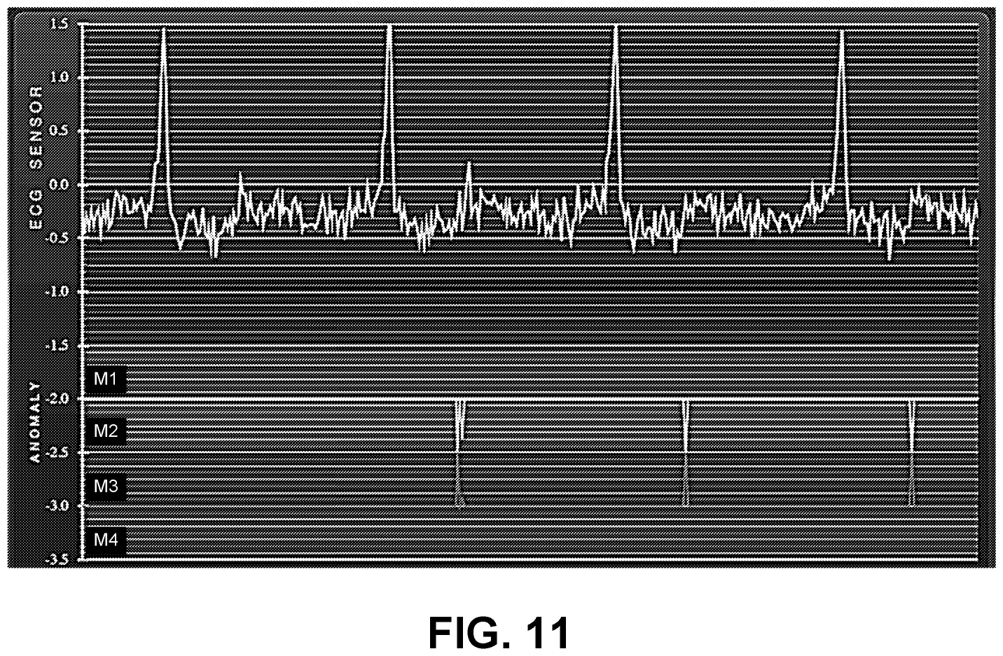

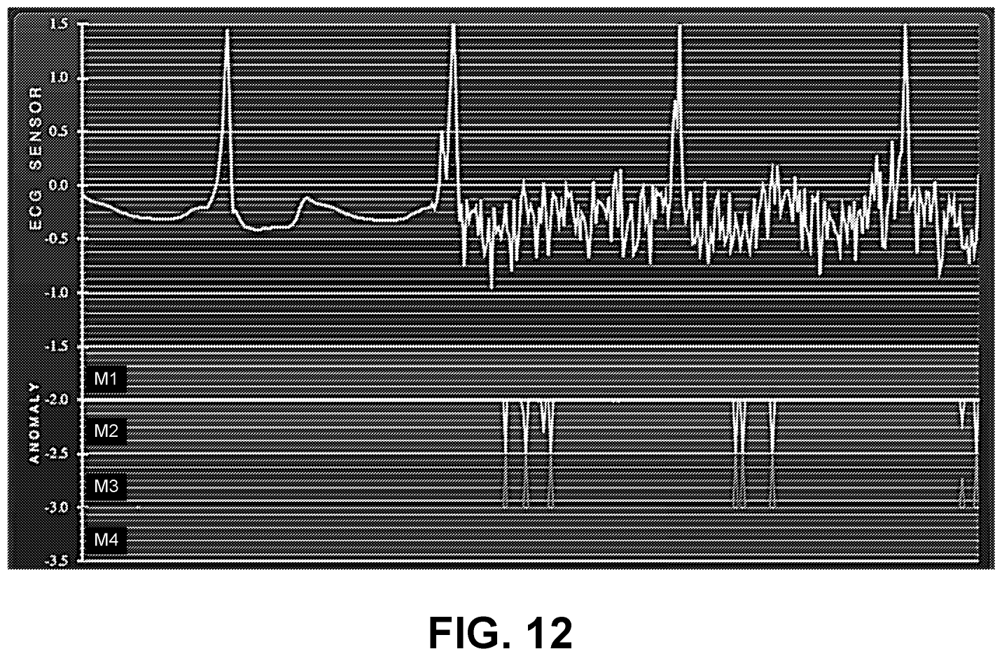

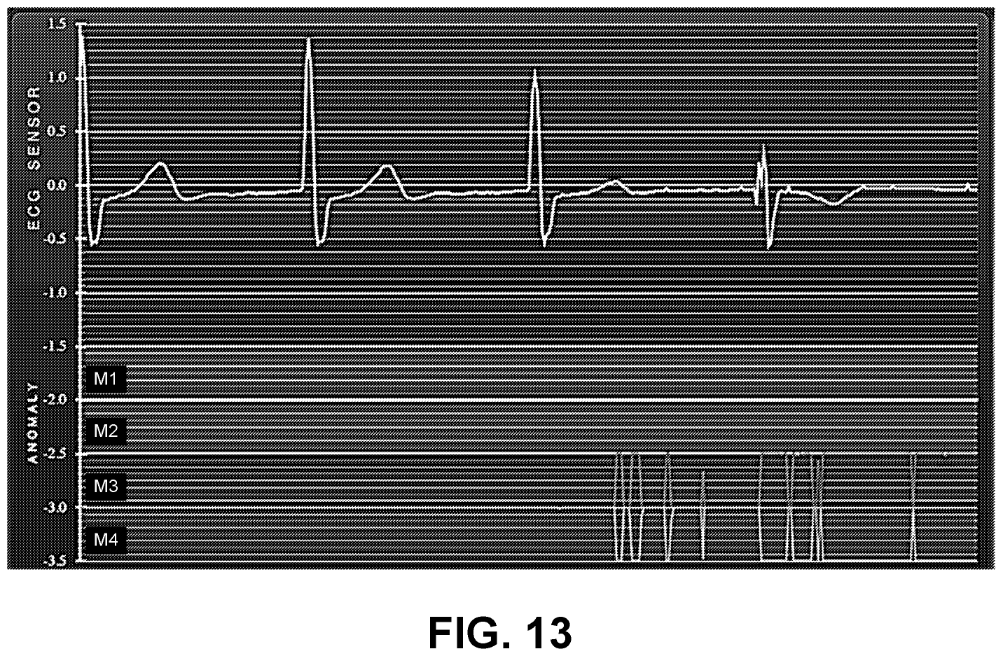

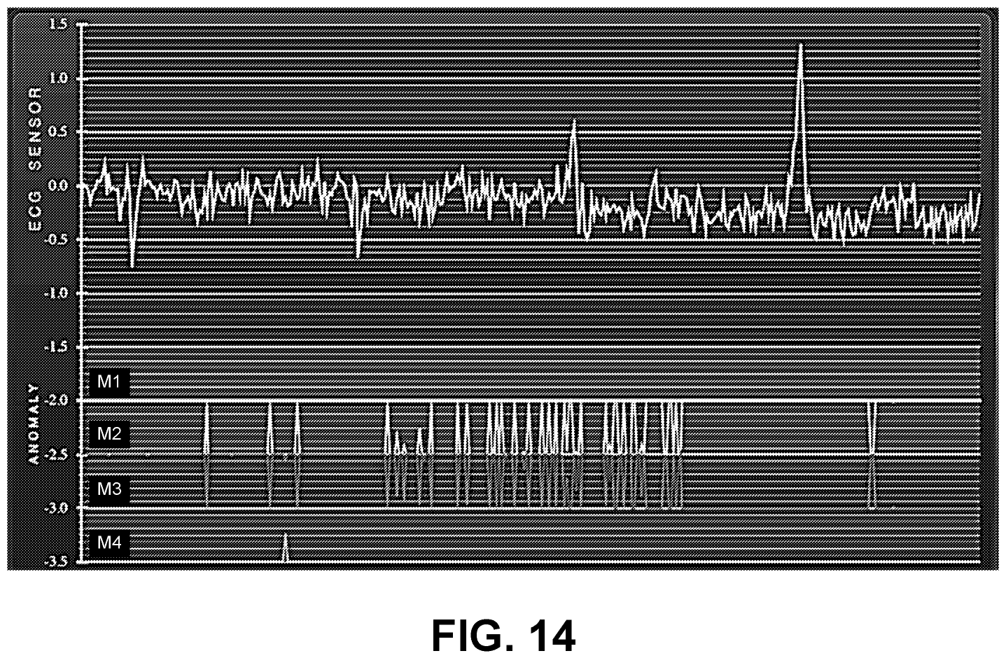

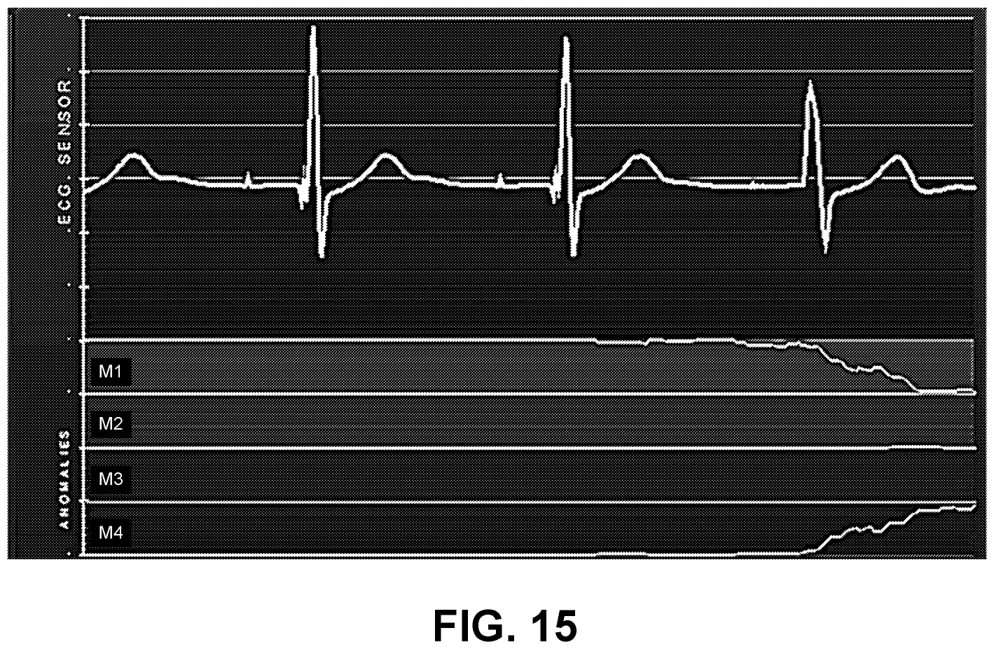

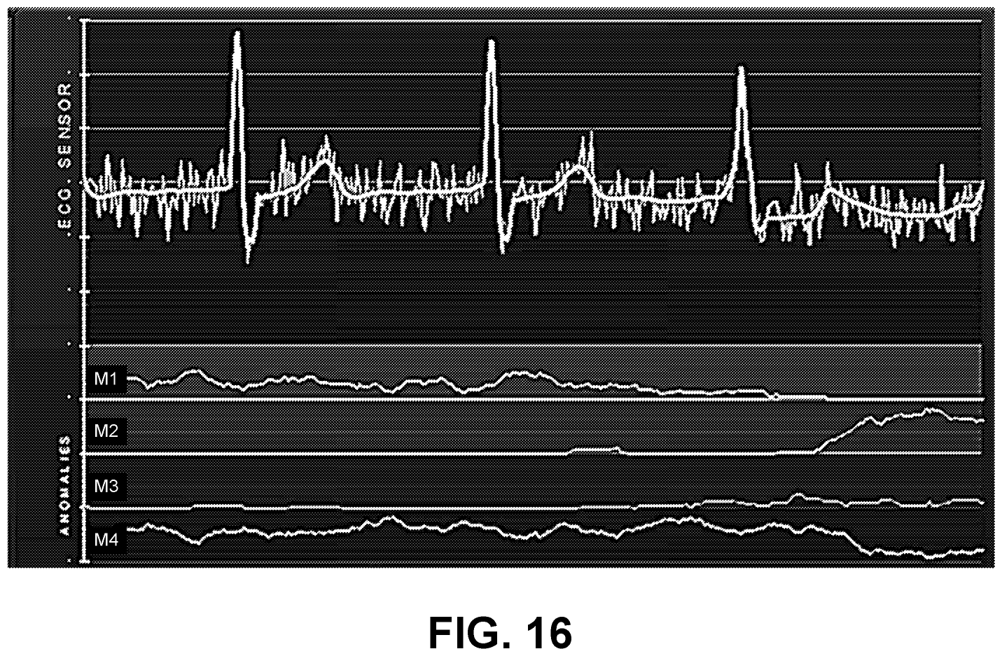

The following figures illustrate examples of the preliminary anomaly detection performance for various scenarios:
FIG. 10 shows single ECG motif and associated ECG raw data streams generated by the ORACLE without any added noise. The ORACLE Anomaly Detection of FIG. 10 illustrates the classification of motif M4 (M4 curve at 100%, all other three curves at 0%) in the absence of noise, i.e., pristine ECG raw data drawn from the currently active ECG motif M4.
FIGS. 11 and 12 show single ECG motif and associated ECG raw data streams generated by the ORACLE with various degrees of super-imposed Gaussian noise. The ORACLE Anomaly Detection of FIG. 11 illustrates the classification of motif M2 (M2 curve at 100%, all other three curves at 0%) in the presence of noise, i.e., ECG raw data stream super-imposed with values drawn from a Gaussian distribution with user-defined standard deviation of 0.10. The ORACLE Anomaly Detection of FIG. 12 illustrates the classification of motif M2 (M2 curve at 100%, all other three curves at 0% with the exception of a few spikes) in the presence of noise, i.e., ECG raw data stream super-imposed with values drawn from a Gaussian distribution with user-defined standard deviation of 0.22.
FIG. 13 shows transition between two ECG motifs without any added noise. The ORACLE Anomaly Detection of FIG. 13 illustrates the classification of transition between motif M4 (M4 curve) and motif M3 (M3 curve) in the absence of noise.
FIG. 14 shows transition between two ECG motifs with superimposed Gaussian noise. The ORACLE Anomaly Detection of FIG. 14 illustrates the classification of transition between motif M3 (M3 curve) and motif M2 (M2 curve) in the presence of noise, i.e., ECG raw data stream super-imposed with values drawn from a Gaussian distribution with user-defined standard deviation of 0.10.
FIG. 15 shows transition between two ECG motifs without any added noise. The ORACLE Anomaly Detection, using a window-averaging approach for the output of the multi-laver neural network, of FIG. 15 illustrates the classification of transition between motif M4 (M4 curve) and motif M3 (M3 curve) in the absence of noise.
FIG. 16 shows transition between two ECG motifs with superimposed Gaussian noise. The ORACLE Anomaly Detection, using a window-averaging approach for the output of the multi-laver neural network, of FIG. 16 illustrates the classification of transition between motif M3 (M3 curve) and motif M2 (M2 curve) in the presence of noise, i.e., ECG raw data stream super-imposed with values drawn from a Gaussian distribution with user-defined standard deviation of 0.10.

The observed spiking in the ORACLE Anomaly Detection performance, especially during motif transition/morphing, may be attributed to a potential overfitting of the multi-layer perceptron (FIG. 7B) used for the classification, especially given the pristine classification of a single motif in the absence of noise (FIG. 10). On the other hand, because the transition between motifs is of importance for an overarching PHM-based health monitoring system for an environment such as a space habitat (and for crew member health status as well), the response of the multi-layer perceptron during the transition should also be considered. A running average can be employed for each motif-identifying curve to address the spiking (FIG. 15 and FIG. 16), but attention will be given towards the resulting/remaining predictive nature of the anomaly curves.

Performance of different network architectures can be evaluated to identify which architecture provides the most useful information, from a PHM point of view, about the transitions/morphing between motifs to facilitate and to contribute to an overarching early warning system for an environment (e.g., space habitat) as well as for the individual or crew member health status. This is a consideration for a PHM for Astronauts paradigm for long duration space missions. The ORACLE can operate as a standalone application or can be queried as an embeddable dynamically linked library module for integration as a subsystem into an overarching PHM-based anomaly detection framework for a wide range of environmental situations.

Verification & validation are system engineering steps to determine if a system or capability satisfies its operational level requirements and can be implemented with an acceptable level of confidence. The capabilities described in this disclosure have demonstrated that, in a realistic environment, levels of uncertainty should be identified and accounted for to develop an integrated capabilities architecture. For example, data involving human crew and related subsystems that are available from environments such as, e.g., submarine, diving, aviation, or space station (e.g., MIR and ISS) can be used to identify uncertainties that are present in different situations. Motif data may be obtained (e.g., in recorded forms) and combined with appropriate habitat system degradation situations and associated recorded data to develop the overall system capabilities. Data from modeling & simulation and/or hybrid environments may be used for development and verification & validation purposes (i.e., “Digital Twin” approach commonly used in the PHM field).

Comparing baseline biometrics across multiple crew members as a normal for various nominal environmental state conditions can be used as a trending approach. This may facilitate the implementation of this overall approach. A digital twin approach, often applied in PHM, using both habitat subsystems and the habitat crew, can be used in realizing this “crew as a sensor” paradigm as an enhancement of an overarching space habitat systems PHM framework.

### Crew as Biosensor to Monitor Environmental Health

Utilizing individuals as biosensors to complement abiotic sensor arrays provides autonomous systems a more comprehensive amount of data to interpret, ensuring the health and safety of the environment. To that effect, each individual can act as a reactive biosensor through autonomic nervous system responses to environmental stimuli, to be quantified by, e.g., ECG measurements, blood oxygenation measurements, ocular structure examination, visual performance assessment, or other biosensor measurements. The ORACLE can be applied to these measurements as previously described.

ECG. Measurable cardiovascular system response is a direct consequence of autonomic nervous system (ANS) stressors. The sympathetic and parasympathetic nervous system (SNS and PSNS), divisions of the ANS, are triggered from perceived and physical stimuli, including changing atmospheric conditions such as lack of O₂ (potentially leading to hypoxia, i.e deficiency in the amount of oxygen reaching the tissues), excessive amounts of O₂ (potentially leading to hyperoxia, i.e., excessive oxygen supply), or excessive amounts of CO₂ (potentially leading to hypercapnia, i.e., excessive carbon dioxide in the bloodstream). The ANS innervates the heart to cope with altered air qualities and atmospheric gas concentrations.

These innervations after the working cycle of the heart through electrophysiological stimulation. It is these electrophysiological stimulations that an ECG measures and records for use in diagnostics of the heart by clinicians. Looking at the impact of O₂, CO₂, CO, and radiation on a standard ECG reading can provide the data for the purpose of biosensing.

Oxygen (O₂) Deprivation (Hypoxia), in a study of 76 normal young adults where arterial oxygen saturation is decreased inducing anoxemia, ECG readings of subjects typically demonstrated cardiovascular distress. The rate of RS-T segment deviation was inversely proportional to arterial oxygen saturation. As oxygen saturation decreased towards 70% within the confines of the study, there was an increasing frequency of RS-T segment elevation or depression amounting to 1 mm across all leads. In nearly every subject, the T-wave of the ECG recordings decreased with diminishing arterial oxygen saturation. With progressive anoxemia, T-waves tended to become inverted. Furthermore, ten subjects exhibited elevation of the P-wave with anoxemia. Readings were taken at the end of ten-minute periods of anoxemia. Low blood oxygen levels may result in shortness of breath, dizziness, visual disorders, and a rapid heartbeat.

Oxygen (O₂) Surplus (Hyperoxia). Hyperoxia effects on ECG have currently only been performed on mice due to cardiotoxic effects of prolonged duration of elevated oxygen inhalation and the risk it poses to humans. Mice exposed to 72 hours of hyperoxia treatment experienced heart arrhythmias compared to the normoxia control group as examined by ECG recordings. Significant prolongation of RR, PR, and QRS intervals were observed. Arrhythmias included missed beats and slower heart rates in the experimental group.

Note that species-specific differences in physiopathology of murine cardiac function contribute to altered morphology of ECG recordings. Manifestations of arrhythmias are different in mice compared to humans and therefore cannot be used as an analog for human ECG arrhythmia analysis due to hyperoxia. It does, however, provide a baseline for potential indications of hyperoxia in human ECG morphology.

Inhalation of Thirty Percent Carbon Dioxide (CO₂). 17 subjects with no history or evidence of cardiac disease were administered CO₂ by mask for an average period of 38 seconds. Lowering and inversion of P waves was noted in five treatments, as well as an increased T-wave was noted in nine of the patients. The study concluded that sudden, severe blood acidosis (blood acidity) had the biggest effect on altering features of the ECG. ECG changes occurred during the period of CO₂ inhalation (lasting 38 seconds on average) as well as persisted for up to several minutes after CO₂ exposure. Prolonged exposure to elevated CO₂ (hypercapnia) may result in mild symptoms of dizziness, drowsiness, and fatigue. Severe symptoms include coma, irregular heartbeat, muscle twitching, and loss of consciousness.

Moderate to Severe Carbon Monoxide (CO) Poisoning. For 230 patients received at a hospital for the treatment of CO poisoning, a baseline ECG was available for 98% of patients. 30% of patients' ECG recordings following CO exposure exhibited ischemic changes (i.e., restriction of blood and oxygen flow to the body) with ST- or T-wave abnormalities. 41% of patients showed nonspecific ST segment changes. Symptoms of CO exposure include a dull headache, nausea, confusion, blurred vision, loss of consciousness, and potentially death.

Radiation Exposure. 197 women with primary carcinoma of the breast were observed for changes in ECG recordings before and after radiation therapy to treat the malignancies. The study concluded there was a statistically significant increase in T-wave changes after treatment. These changes were correlated to the radiation absorbed by the heart, measured two and six months after treatment with more than 20 Gy (unit, Gray: absorption of one joule of radiation energy per kilogram of matter) administered.

Blood Oxygenation. Pulse oximeters can be utilized to measure arterial oxygen saturation (SpO₂). With the form factor of a small device that fits to, e.g. the tip of the finger, they are typically used in emergency diagnostic techniques. It functions to screen for hypoxemia, i.e., lack of oxygen in the blood, to assist in medical treatment.

For the purpose of monitoring space habitat atmosphere health, SpO₂ readings would function to observe abnormalities in O₂ and CO₂. However, pulse oximeters lack the specificity to accurately determine oxygen saturation in the presence of other atmospheric gasses such as CO. Wien CO binds with hemoglobin (CO has 210 times the affinity for hemoglobin compared to oxygen), SpO₂ measurements consistently overestimate the amount of actual arterial oxygen saturation but can be used as an indicator of environmental changes.

Ocular Structure and Visual Performance. Risks to the ocular system and vision during prolonged stays aboard orbiting or landed space habitats (e.g., NASA Gateway/Artemis programs) around or on the Moon, Mars, or beyond include possible corneal, lens, and retinal damage from exposure to various types of radiation (UV, IR, other high-energy radiation, e.g., due to lack of space habitat shielding), hypoxia, and toxic environmental poisoning (e.g., due to several combustion events that have occurred aboard space stations in the past, such as the one aboard the Mir space station where a fault in a perchlorate canister of a Vika or TGK oxygen generator, also known as Solid Fuel Oxygen Generation (SFOG), exploded in the cabin). As such, the ocular structure and vision, i.e., crew visual performance, may serve as biomarkers that can be used to assess changes in space habitat environments the crew is exposed to. Handheld microscopy, slit-lamp examination, fundoscopy, as well as visual field testing are methods that may be used to assess these biomarkers.

Spaceflight-Associated Neuro-ocular Syndrome (SANS). The Visual Impairment Intracranial Pressure (VIP) syndrome, recently redefined by NASA as the Spaceflight-Associated Neuro-ocular Syndrome (SANS), is evidenced by visual changes in International Space Station (ISS) astronauts. SANS/VIIP is an example of a maladaptive outcome of microgravity such as bone loss and muscle atrophy that may persist long after return to Earth, perhaps permanently. Overall SANS has been identified by NASA as the number one risk for long-duration spaceflight. SANS/VIIP manifests itself as decreased near-field visual acuity. Although the microgravity-induced cephalad fluid shift is considered the primary causative factor, a secondary contributing factor includes elevated cabin CO₂ concentration.

Vascular and Ocular Structure. In addition to microgravity, adverse outgassing events have occurred in the past due to several combustion events aboard space stations, such as the fire that occurred aboard the Mir space station. These events may have an influence on both the ocular structure and associated visual performance. For example, sealed environments like the ISS exhibit elevated CO₂, a potent arteriolar vasodilator that could further affect cerebral blood volume and cerebral blood flow, intracranial compliance, and intracranial pressure. CO₂ is an extremely potent vasodilator, and its levels on the ISS are tenfold Earth's levels. If maintained over an extended period of time, elevated CO₂ could induce remodeling in the vasculature and potentially posterior ocular structures, suggesting a potential environmental change indicator modality.

As such, changes to the ocular system and to the associated visual performance may serve as indicators for environmental changes aboard space habitats, albeit on much larger time scales not suitable for immediate change detection. Ocular and visual performance changes can record subtle drifts/shifts in environmental parameters that may go unnoticed by other, abiotic sensors aboard a space habitat that are either not sensitive enough to detect subtle changes, and/or are geared towards rapid change detection only.

With reference to FIG. 17, shown is a schematic block diagram of a computing (or processing) device 1500 that can be utilized for implementing the ORACLE application or embeddable dynamically linked library module using the described techniques. In some embodiments, among others, the computing device 1500 may represent a mobile device (e.g. a smartphone, tablet, computer, etc.) or other processing device. Each computing device 1500 includes processing circuitry comprising at least one processor circuit, for example, having a processor 1503 and a memory 1506, both of which are coupled to a local interface 1509. To this end, each computing device 1500 may comprise, for example, at least one server computer or like device. The computing device 1500 can comprise one or more computing devices configured for distributed processing. The local interface 1509 may comprise, for example, a data bus with an accompanying address/control bus or other bus structure as can be appreciated.

In some embodiments, the computing device 1500 can include one or more network interfaces 1510. The network interface 1510 may comprise, for example, a wireless transmitter, a wireless transceiver, and a wireless receiver. As discussed above, the network interface 1510 can communicate to a remote computing device using a Bluetooth protocol. As one skilled in the art can appreciate, other wireless protocols may be used in the various embodiments of the present disclosure. The computing device 1500 can be communicatively coupled to one or more user devices (e.g., wrist-worn sensor or smartphone, etc.) allowing for communication of results back to the user.

Stored in the memory 1506 are both data and several components that are executable by the processor 1503. In particular, stored in the memory 1506 and executable by the processor 1503 are the ORACLE application or embeddable dynamically linked library module 1515, application program 1518, and potentially other applications. For example, the ORACLE application or embeddable dynamically linked library module 1515 can be implemented within a construction performance modeling and simulation (CPMS) framework. Also stored in the memory 1506 may be a data store 1512 and other data. In addition, an operating system may be stored in the memory 1506 and executable by the processor 1503.

It is understood that there may be other applications that are stored in the memory 1506 and are executable by the processor 1503 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1506 and are executable by the processor 1503. In this respect, the term “executable” means a program, application or module file that is in a form that can ultimately be run by the processor 1503. Examples of executable programs, applications or modules may be, for example, a compiled program that can be translated into machine code in a format that can be loaded into a random access portion of the memory 1506 and run by the processor 1503, source code that may be expressed in proper format such as object code that is capable of being loaded into a random access portion of the memory 1506 and executed by the processor 1503, or source code that may be interpreted by another executable program, application or module to generate instructions in a random access portion of the memory 1506 to be executed by the processor 1503, etc. An executable program, application or module may be stored in any portion or component of the memory 1506 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1506 is defined herein as including both volatile and nonvolatile memory and data storage components. Volatile components are those that do not retain data values upon loss of power. Nonvolatile components are those that retain data upon a loss of power. Thus, the memory 1506 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1503 may represent multiple processors 1503 and/or multiple processor cores and the memory 1506 may represent multiple memories 1506 that operate in parallel processing circuits, respectively. In such a case, the local interface 1509 may be an appropriate network that facilitates communication between any two of the multiple processors 1503, between any processor 1503 and any of the memories 1506, or between any two of the memories 1506, etc. The local interface 1509 may comprise additional systems designed to coordinate this communication, including, for example, performing load balancing. The processor 1503 may be of electrical or of some other available construction.

Although the ORACLE application or embeddable dynamically linked library module 1515 and the application program 1518, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the ORACLE application or ernbeddable dynamically linked library rnodule 1515 and the application program 1518, that comprises software or code can be embodied in any non-transitory computer-readable medium for use by or in connection with an instruction execution system such as, for example, a processor 1503 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the ORACLE application or embeddable dynamically linked library module 1515 and the application program 1518, may be implemented and structured in a variety of ways. For example, one or more applications described may be implemented as modules or components of a single application. For example, separate applications can be executed for the similarity and compatibility checking and management workflows as illustrated in FIGS. 3 and 7. Further, one or more applications described herein may be executed in shared or separate computing devices or a combination thereof. For example, a plurality of the applications described herein may execute in the same computing device 1500, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method for anomaly detection in sensor data, comprising:
applying data from portions of a real-time sensor signal to an artificial neural network trained to identify a plurality of motifs associated with the real-time sensor signal based upon corresponding output signals of the artificial neural network, the corresponding output signals providing an indication of correlation of the real-time sensor signal to each of the plurality of motifs;
detecting a transition from a first motif of the plurality of motifs to a second motif of the plurality of motifs based upon changes in a first output signal corresponding to the first motif and a second output signal corresponding to the second motif; and
identifying a change in an environmental condition based upon the transition between the first and second motifs.

2. The method of claim 1, wherein the artificial neural network is a multilayer feedforward network, a deep learning network, a convolutional neural network, or a recursive neural network.

3. The method of claim 1, wherein the artificial neural network is trained using error-backpropagation.

4. The method of claim 1, wherein the artificial neural network is trained using a stochastic training approach.

5. The method of claim 1, wherein the artificial neural network is trained using data generated from previously extracted motifs associated with the real-time sensor signal.

6. The method of claim 5, wherein the generated data comprises transitions between two of the previously extracted motifs.

7. The method of claim 6, wherein the generated data comprise superimposed noise.

8. The method of claim 5, wherein at least one of the previously extracted motifs comprises superimposed noise.

9. The method of claim 5, wherein the previously extracted motifs are generated by averaging raw sensor signal data and binning into a histogram.

10. The method of claim 1, wherein the real-time sensor signal is a biotic signal.

11. The method of claim 10, wherein the biotic signal is a vital sign of an individual exposed to the environmental condition.

12. The method of claim 11, wherein the biotic signal is an electrocardiogram (ECG) signal.

13. The method of claim 11, wherein the biotic signal is blood oxygenation, EEGs (brain waves), temperature, ocular structure changes, or visual field changes.

14. The method of claim 11, wherein the environmental condition is associated with a habitat of the individual.

15. The method of claim 1, wherein the real-time sensor signal is an abiotic signal.

16. A method for generating training data for training a network, comprising:
selecting a plurality of motifs associated with a desired training signal;
generating the desired training signal by transitioning between different motifs of the plurality of motifs in a pseudo-random basis; and
generating training data sets from the desired training signal.

17. The method of claim 16, wherein the transitions between different motifs comprise nonlinear transitions.

18. The method of claim 16, wherein generating the desired training signal further comprises superimposing noise.

19. The method of claim 16, wherein the plurality of motifs are generated by averaging raw sensor signal data and binning into a histogram.

20. The method of claim 16, further comprising training an artificial neural network using at least a portion of the generated training data sets.
